(19) United States
(12) Patent Application Publication
(10) Pub. No.: US 2022/0379088 A1
(43) Pub. Date: Dec. 1, 2022

(54) CATHETER ASSEMBLY HAVING A SEPTUM

(71) Applicant: Becton, Dickinson and Company, Franklin Lakes, NJ (US)

(72) Inventors: Megan Scherich, Salt Lake City, UT (US); John Lackey, West Valley City, UT (US); Curtis H. Blanchard, Riverton, UT (US); BreAnna Lackey, West Valley City, UT (US); Yiping Ma, Layton, UT (US); Shaun Staley, Murray, UT (US); Jonathan Karl Burkholz, Salt Lake City, UT (US); Shaun Lauer, West Jordan, UT (US); Thomas Solosko, Draper, UT (US)

(21) Appl. No.: 17/737,416

(22) Filed: May 5, 2022

Related U.S. Application Data
(60) Provisional application No. 63/192,977, filed on May 25, 2021.

(51) Int. Cl. A61M 25/00; A61M 25/06; A61M 39/06

(52) U.S. Cl. CPC A61M 25/0097; A61M 25/0606; A61M 39/0606; A61M 25/0631; A61M 2039/0036

(57) ABSTRACT

A catheter assembly may include a catheter adapter, which may include a body and a side port extending outwardly from the body. The body may include a distal end, a proximal end, and an inner surface extending through the distal end and the proximal end and forming a lumen. The catheter assembly may include one or more components to facilitate flushing of the catheter assembly. For example, an angle of the side port with respect to a longitudinal axis of the body may be adjustable. The catheter assembly may include a catheter extending distally from the distal end of the catheter adapter. The catheter assembly may include a septum disposed within the lumen proximal to a side port pathway extending through the side port and in fluid communication with the lumen.

### RELATED APPLICATIONS

This application claims the benefit of U.S. Provisional Patent Application No. 63/192,977, filed on May 25, 2021, entitled CATHETER ASSEMBLY HAVING A SEPTUM, which is incorporated herein in its entirety.

### BACKGROUND

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of a catheter assembly that includes the catheter. After placement of the needle has been confirmed, the clinician may remove the needle, leaving the catheter in place for future blood withdrawal or fluid infusion.

Many catheter assemblies have a septum proximal to an extension tube inlet, creating a region distal to the septum that is difficult to flush free of fluids (such as blood or infusates). Stagnant fluid within the region distal to the septum may lead to accumulation of bacteria within the catheter assembly, which may result in infection or removal of the catheter from the patient.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices. The present disclosure relates more particularly to a catheter assembly that includes a septum. In some embodiments, the septum may facilitate flushing and/or decrease a risk of thrombus of the integrated catheter assembly.

In some embodiments, the catheter assembly may include catheter adapter, which may include a body and a side port extending outwardly from the body. In some embodiments, the body may include a distal end, a proximal end, and an inner surface extending through the distal end of the body and the proximal end of the body. In some embodiments, the inner surface may form a lumen. In some embodiments, an angle of the side port with respect to a longitudinal axis of the body may be less than 45°. In some embodiments, the angle of the side port with respect to the longitudinal axis of the body may be about 25°.

In some embodiments, the catheter assembly may include a catheter, which may extend distally from the distal end of the catheter adapter. In some embodiments, the catheter may include a peripheral intravenous catheter, a peripherally-inserted central catheter, or a midline catheter. In some embodiments, the septum may be disposed within the lumen. In some embodiments, the septum may be disposed proximal to a side port pathway, which may extend through the side port and in fluid communication with the lumen. In further detail, in some embodiments, at least a portion of the septum may be disposed proximal to the side port pathway.

In some embodiments, the catheter adapter may include a region in which fluid may tend to become stagnant. In some embodiments, the region may include a distal face of the septum and an area within the lumen adjacent to the septum. Stagnant fluid within the region may lead to an accumulation of bacteria within the catheter assembly, which may result in infection or removal of the catheter from the patient.

In some embodiments, a distal end of the septum may be aligned with or distal to a proximal edge of the side port pathway, which may decrease a size of a region and decrease a risk of stagnant fluid and accumulation of bacteria in the region. In some embodiments, the septum may extend distal to the side port pathway. In these embodiments, the septum may include a tunnel extending therethrough and aligned with the side port pathway. In some embodiments, the side port pathway may be generally cylindrical. In some embodiments, the distal end of the septum may be aligned with the proximal edge of the side port pathway. In some embodiments, the distal end of the septum may be distal to the proximal edge of the side port pathway. In some embodiments, a side of the septum closest to the side port pathway may be aligned with or distal to a proximal edge of the side port pathway. In some embodiments, the side of the septum closest to the side port pathway may be aligned with the proximal edge of the side port pathway.

In some embodiments, the distal end of the septum may be asymmetric, which may decrease a risk of stagnant fluid and accumulation of bacteria in the region. In some embodiments, the distal end of the septum may extend across a width of the lumen. In some embodiments, the distal end of the septum may include a first side and a second side. In some embodiments, the distal end may be angled between the first side and the second side. In some embodiments, the first side may be on a same side of the lumen as the side port. In some embodiments, the second side may extend distal to the first side.

In some embodiments, the distal end of the septum may include an arm extending distally and contacting the inner surface. In some embodiments, the arm may be on an opposite side of the lumen as the side port. In some embodiments, a distal end of the arm may include an extension extending inwardly. In some embodiments, the arm may be configured to direct fluid flow to the region. In some embodiments, a proximal side of the extension may be disposed distal to a proximal edge of the side port pathway.

In some embodiments, a portion of the inner surface proximal to the catheter and distal to the septum may include a protrusion extending inwardly into the lumen opposite the side port pathway. In some embodiments, the protrusion may include a ramp in a proximal direction. In some embodiments, a proximal end of the ramp may be proximate the septum. In some embodiments, the catheter assembly may include a wedge securing the catheter within the distal end of the catheter adapter. In some embodiments, a distal end of the protrusion may be proximate the wedge. In some embodiments, the proximal end of the ramp may be aligned with a wall of the catheter. In some embodiments, a proximal end of the ramp may be proximate the septum. In some embodiments, a distal face of the septum may include a protrusion. In some embodiments, the proximal end of the ramp may contact the protrusion.

In some embodiments, a distal portion of the protrusion may be parallel to a longitudinal axis of the catheter assembly. In some embodiments, a proximal portion of the protrusion may include a ramp in a distal direction. In some embodiments, a proximal end of the catheter may be bonded with the catheter adapter. In some embodiments, the distal portion may be aligned with a wall of the catheter. In these and other embodiments, a proximal end of the catheter may be bonded with the catheter adapter.

In some embodiments, the portion of the inner surface proximal to the catheter and distal to the septum may include an annular shoulder. In some embodiments, the annular shoulder may be disposed at a 90-degree angle. In some embodiments, the catheter assembly may include the wedge securing the catheter within the distal end of the catheter adapter, and the shoulder may be proximate a proximal end of the wedge. In some embodiments, a distal end of the septum may be aligned with or distal to the side port pathway.

In some embodiments, a method may include injecting a curable material into the catheter adapter of the catheter assembly. In some embodiments, the curable material may be injected between a proximal end of the catheter and a distal end of the septum. In some embodiments, the method may include creating a fluid pathway through the curable material, which may include flushing the catheter assembly through the side port pathway. In some embodiments, the method may include inserting an introducer needle (or another cannula or mandrel) through the curable material. In some embodiments, the method may include curing the curable material after creating the fluid pathway and inserting the introducer needle through the curable material. In some embodiments, the curable material may be cured via heat, ultraviolet light, or another external energy source. In some embodiments, the curable material may include silicone, foam, or gel.

In some embodiments, the catheter assembly may include an asymmetric insert disposed between the septum and a proximal end of the catheter. In some embodiments, the asymmetric insert may include an opening extending therethrough and aligned with a longitudinal axis of the catheter assembly. In some embodiments, the catheter assembly may include a seal component proximal to the side port pathway and distal to the septum. In some embodiments, the seal component may be configured to move from an open position to a closed position in response to withdrawal of the introducer needle. In some embodiments, the catheter assembly may include a spring coupled to the seal component and configured to bias the seal component in the closed position.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a cross-sectional view of an example catheter assembly, according to some embodiments;

FIG. 2A is an upper perspective view of the catheter assembly, illustrating an example side port at an example angle, according to some embodiments;

FIG. 2B is an upper perspective view of the catheter assembly, illustrating the side port at another example angle, according to some embodiments;

FIG. 3 is a cross-sectional view of the catheter assembly, illustrating an example septum, according to some embodiments;

FIG. 4 is a cross-sectional view of the catheter assembly, illustrating the septum including an example tunnel, according to some embodiments;

FIG. 5 is a cross-sectional view of the catheter assembly, illustrating the septum including an example angled distal face, according to some embodiments;

FIG. 6 is a cross-sectional view of the catheter assembly, illustrating the septum including an example arm, according to some embodiments; and

FIG. 7 is a cross-sectional view of the catheter assembly, illustrating the septum including the arm and an example extension from the arm, according to some embodiments.

FIG. 8A is a cross-sectional view of the catheter assembly, illustrating an example protrusion, according to some embodiments;

FIG. 8B is a cross-sectional view of the catheter assembly, illustrating the protrusion according to some embodiments;

FIG. 8C is a cross-sectional view of the catheter assembly, illustrating the protrusion according to some embodiments;

FIG. 8D is a cross-sectional view of the catheter assembly, a distal end of an example septum aligned with an example side port pathway, according to some embodiments;

FIG. 9A is a cross-sectional view of the catheter assembly, illustrating an example curable material, according to some embodiments;

FIG. 9B is a cross-sectional view of the catheter assembly, illustrating the curable material in response to flushing, according to some embodiments; and

FIG. 9C is a cross-sectional view of the catheter assembly, illustrating an example introducer needle extending through the curable material, according to some embodiments.

FIG. 10 is a cross-sectional view of the catheter assembly, illustrating an example insert, according to some embodiments;

FIG. 11 is a cross-sectional view of the catheter assembly, illustrating another example insert, according to some embodiments;

FIG. 12A is a cross-sectional view of the catheter assembly, illustrating an example seal component in an example open position, according to some embodiments; and

FIG. 12B is a cross-sectional view of the catheter assembly, illustrating the seal component in an example closed position, according to some embodiments.

### DESCRIPTION OF EMBODIMENTS

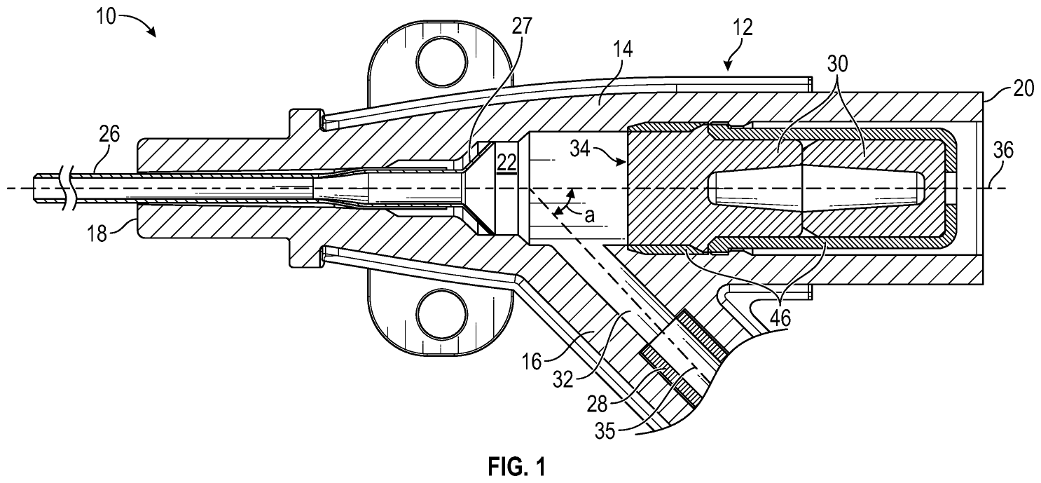

Referring now to FIG. 1, a catheter assembly 10 is illustrated. In some embodiments, the catheter assembly 10 may include a catheter adapter 12, which may include a body 14 and a side port 16 extending outwardly from the body 14. In some embodiments, the body 14 may include a distal end 18, a proximal end 20, and an inner surface 22 forming a lumen 24. In some embodiments, the lumen 24 may extend through the distal end 18 and the proximal end 20.

In some embodiments, the catheter assembly 10 may include a catheter 26 extending distally from the distal end 18 of the body 14. In some embodiments, the catheter 26 may be secured within the body 14. In some embodiments, the catheter 26 may include a peripheral intravenous catheter, a peripherally-inserted central catheter, or a midline catheter. In some embodiments, a needle hub may be coupled to an introducer needle (not illustrated), which may extend through the catheter 26 and may be removed after the catheter 26 is placed within vasculature of a patient. In some embodiments, the catheter 26 may be secured within the catheter adapter 12 by a wedge 27.

In some embodiments, a distal end of an extension tube 28 may be integrated with the side port 16. In some embodiments, a proximal end of the extension tube 28 may be coupled to an infusion device, a flush device, or a blood collection device. In some embodiments, the catheter adapter 12 may include a septum 30 disposed within the lumen 24 proximal to the side port 16 and/or a side port pathway 32 extending through the side port 16 and in fluid communication with the lumen 24. In further detail, in some embodiments, at least a portion of the septum 30 may be disposed proximal to the side port 16 and/or the side port pathway 32. In some embodiments, the catheter adapter 12 may include a region 34 in which fluid may tend to become stagnant, despite flushing through the side port 16. In some embodiments, the region 34 may include or correspond to a dead space. In some embodiments, the region 34 may include a distal face of the septum 30 and an area within the lumen 24 adjacent thereto. Stagnant fluid within the region 34 may lead to occlusion or an accumulation of bacteria within the catheter assembly 10, which may result in infection or removal of the catheter 26 from the patient.

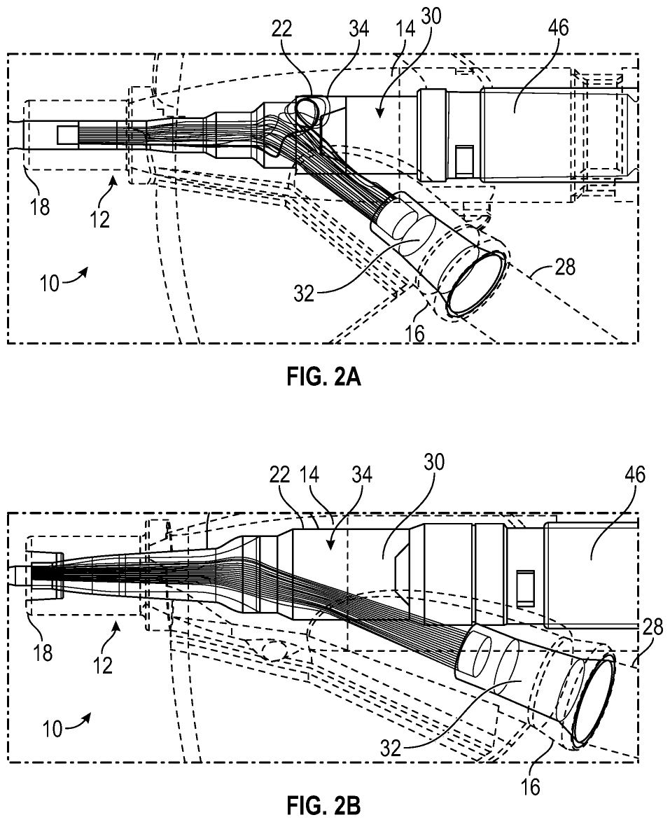

In some embodiments, an angle a of the side port 16 (which may be measured by a central axis 35 of the side port 16) with respect to a longitudinal axis 36 of the catheter adapter 12 may be less than 45°, which may facilitate insertion of a probe distally through the side port 16 and/or the catheter 26. In some embodiments, the angle a that is less than 45° may enhance a tendency of fluid to become stagnant in the region 34. In these and other embodiments, the septum 30 may be configured to facilitate and increase flushing of the region 34. Referring now to FIG. 2A, the angle a of the side port 16 is illustrated at 45°, which may direct fluid towards the region 34 to facilitate flushing. Referring now to FIG. 2B, the angle a of the side port 16 is illustrated at 25°, which may facilitate less bending and enhanced insertion of a probe as it moves distally through the side port 16 and/or through the catheter 26. However, when the angle a is less than 45°, less fluid may be directed to the region 34.

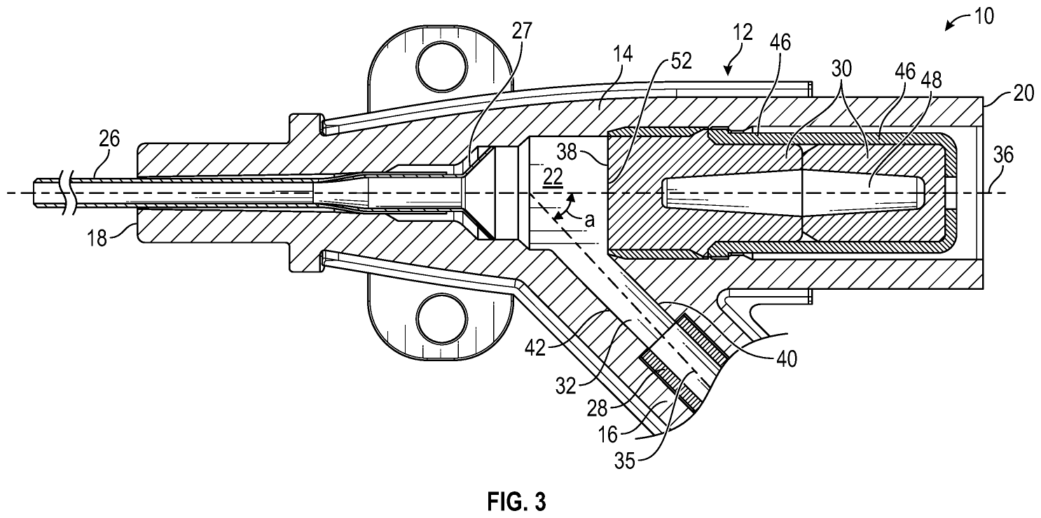

Referring now to FIG. 3, in some embodiments, the septum 30 may be elongated or moved distally to eliminate or decrease a size of the region 34 (see, for example, FIG. 1) and a tendency of fluid to become stagnant within the catheter adapter 12. As illustrated in FIG. 3, in some embodiments, a distal end 38 of the septum 30 may be aligned with or nearly or approximately aligned with a proximal edge 40 of the side port pathway 32, which may be opposite a distal edge 42 of the side port pathway 32. In some embodiments, the distal end 38 may be distal to the proximal edge 40. In some embodiments, the septum 30 aligned with (or nearly or approximately aligned with) or distal to the proximal edge 40 may prevent fluid from flowing proximal to the proximal edge 40 within the lumen 24.

In some embodiments, the septum 30 may include an elastomeric body 44 and/or a canister 46. In some embodiments, all or a portion of the elastomeric body 44 may be disposed within the canister 46, which may be constructed of metal or another suitable material. In some embodiments, the canister 46 may facilitate securement of the septum 30 within the lumen 24. In some embodiments, the septum 30 may not include the canister 46. In some embodiments, the septum 30 may include a two-piece low drag septum with a cavity 48 disposed in a middle of the septum 30 to decrease drag on the introducer needle, which may be proximally withdrawn through the septum 30. In other embodiments, the septum 30 may include a single piece. In some embodiments, a shape of the septum 30 may vary.

In some embodiments, the angle a may be less than 45°, such as, for example, 25°, in order to facilitate probe insertion, which may decrease flushability of the catheter adapter 12, but the septum 30 may increase flushability of the catheter adapter 12 by decreasing the region 34 (see, for example FIG. 1) of an area in which fluid may become stagnant. Thus, the catheter assembly 10 may facilitate probe insertion and also flushability, according to some embodiments.

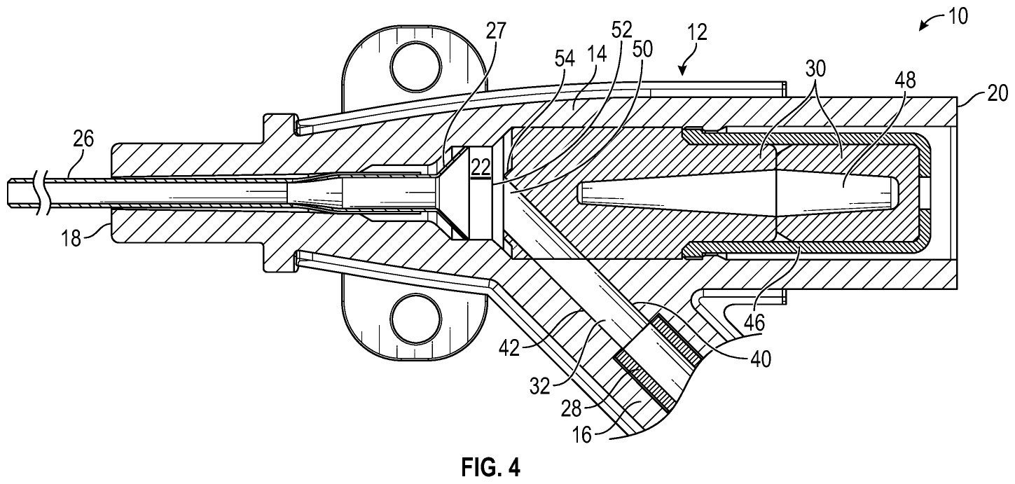

Referring now to FIG. 4, in some embodiments, the septum 30 may extend distal to the proximal edge 40 of the side port pathway 32 or the distal edge 42 of the side port pathway 32, which may decrease a size of the region 34 (see, for example, FIG. 1). In these embodiments, the septum 30 may include a tunnel 50 extending therethrough and aligned with the side port pathway 32. For example, the tunnel 50 and the side port pathway 32 may form a generally straight pathway or a central axis of the tunnel 50 may be aligned with a central axis of the side port 16. In some embodiments, the tunnel 50 may be a partial tunnel or hole extending partially through the septum 30 or a complete tunnel extending entirely through the septum 30.

In some embodiments, the side port pathway 32 and/or the tunnel 50 may be generally cylindrical, which may make the side port pathway 32 and/or the tunnel 50 easier to flush. In some embodiments, the tunnel 50 and/or the side port pathway 32 may have another suitable shape. Referring back to FIGS. 1 and 3, in some embodiments, the distal end 38 of the septum 30 may include a distal face 52 that is generally flat or planar, as illustrated, for example, in FIGS. 1 and 3. Referring back to FIG. 4, in some embodiments, the distal face 52 may be generally flat or planar or may include a protrusion 54, which may decrease a size of the region 34 (see, for example, FIG. 1). In some embodiments, the tunnel 50 may extend through the distal face 52 and/or the protrusion 54.

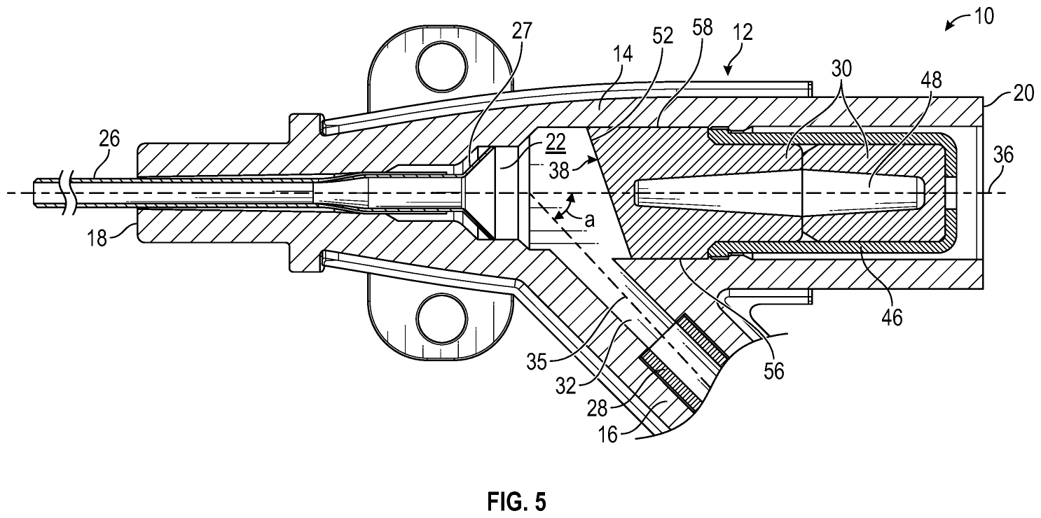

Referring now to FIG. 5, in some embodiments, the distal end 38 of the septum 30 may be asymmetric, which may decrease a risk of stagnant fluid and accumulation of bacteria in within the lumen 24. In some embodiments, the distal end 38 of the septum 30 may include the distal face 52, which may extend across a width of the lumen 24. In some embodiments, the distal face 52 may be generally planar. In some embodiments, the distal face 52 may be angled with respect to the longitudinal axis 36. In some embodiments, the distal end 38 of the septum 30 may include a first side 56 and a second side 58. In some embodiments, the distal face 52 may be angled between the first side 56 and the second side 58. In some embodiments, the first side 56 may be on a same side of the lumen 24 as the side port 16. In some embodiments, the second side 58 may extend distal to the first side 56. In some embodiments, the first side 56 and the second side 58 may contact opposite sides of the inner surface 22.

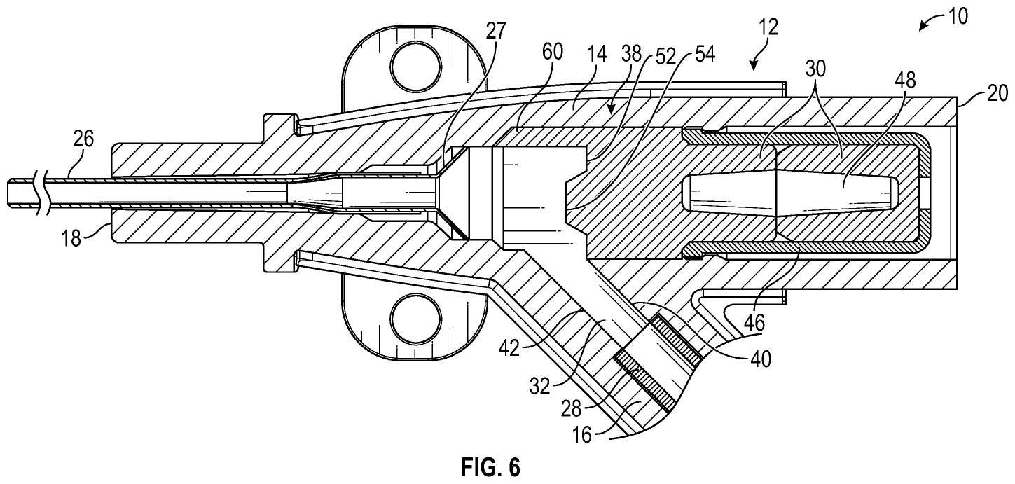

Referring now to FIG. 6, in some embodiments, the distal end 38 of the septum 30 may include an arm 60 extending distally and contacting the inner surface 22. In some embodiments, the arm 60 may extend distally from the distal face 52. In some embodiments, the arm 60 may be on an opposite side of the lumen 24 as the side port 16 to facilitate fluid flow through the side port 16. In some embodiments, the arm 60 may decrease an area of the lumen 24 in which fluid may otherwise become stagnant. In some embodiments, the arm 60 may extend distal to the distal edge 42. In some embodiments, the arm 60 may extend to the wedge 27.

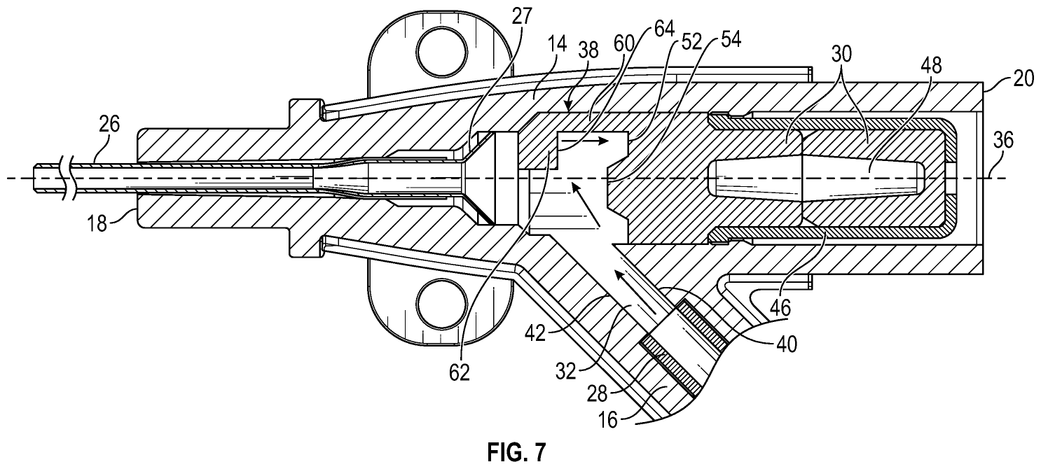

Referring now to FIG. 7, in some embodiments, a distal end of the arm 60 may include an extension 62 extending inwardly. In some embodiments, the extension 62 may extend inwardly toward the longitudinal axis 36. In some embodiments, the extension 62 may extend inwardly to or beyond the longitudinal axis 36. In some embodiments, the arm 60 may be configured to direct fluid flow to the region. In some embodiments, a proximal side 64 of the extension 62 may be disposed distal to the proximal edge 40 of the side port pathway 32. In some embodiments, the arm 60 and/or the extension 62 may be configured to direct fluid proximally towards the distal face 52, which may facilitate flushing.

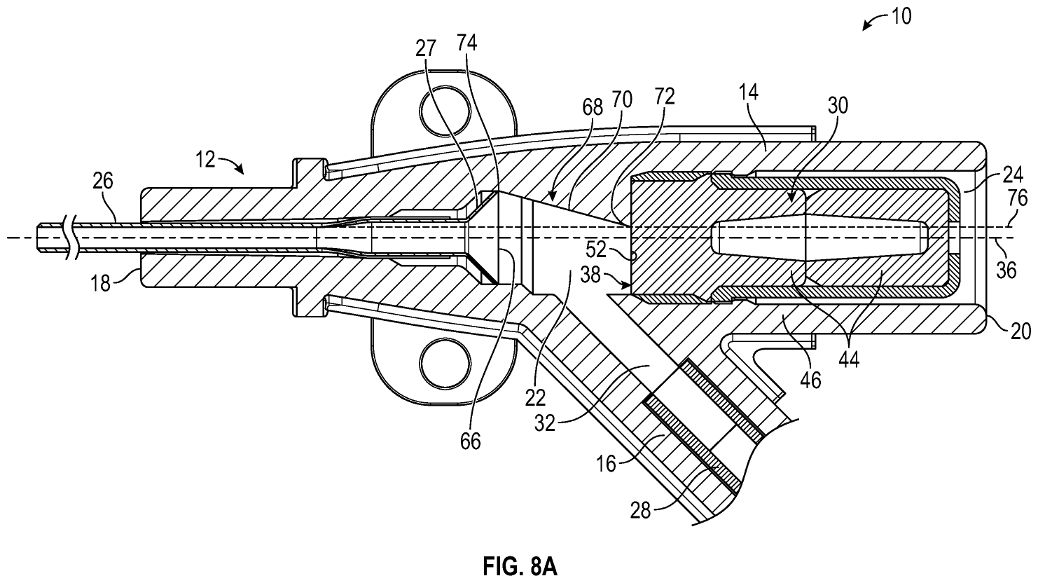

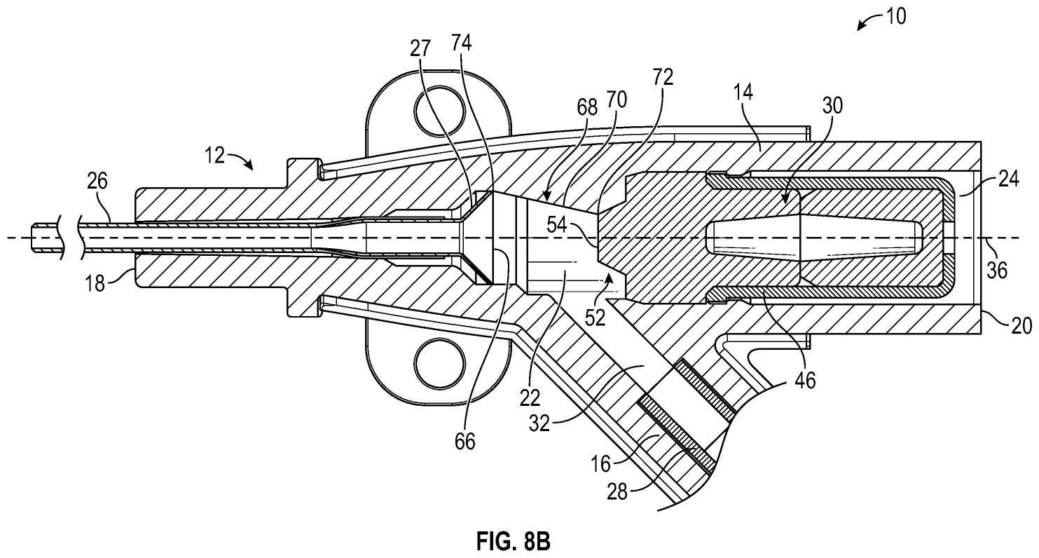

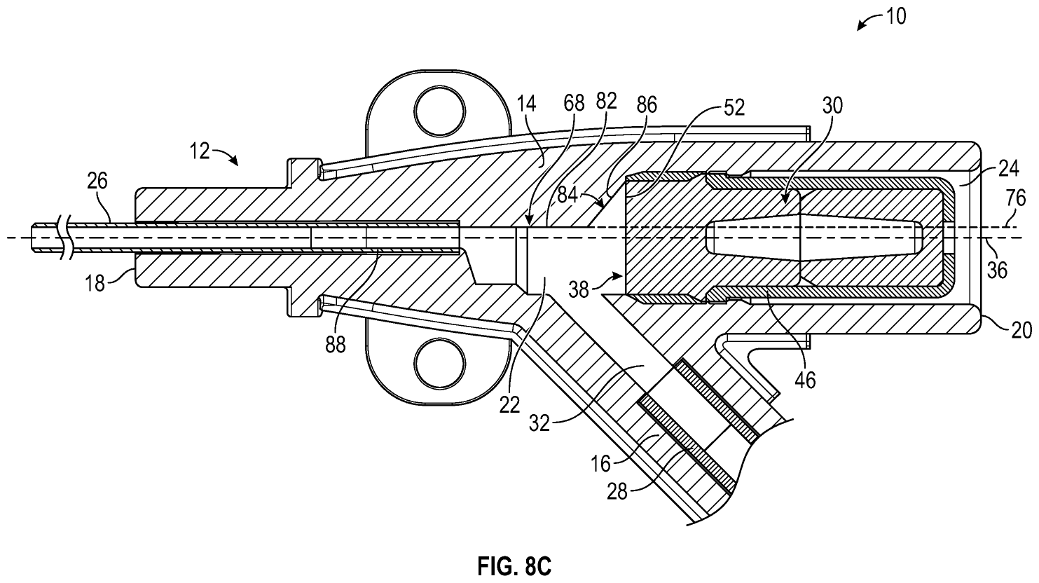

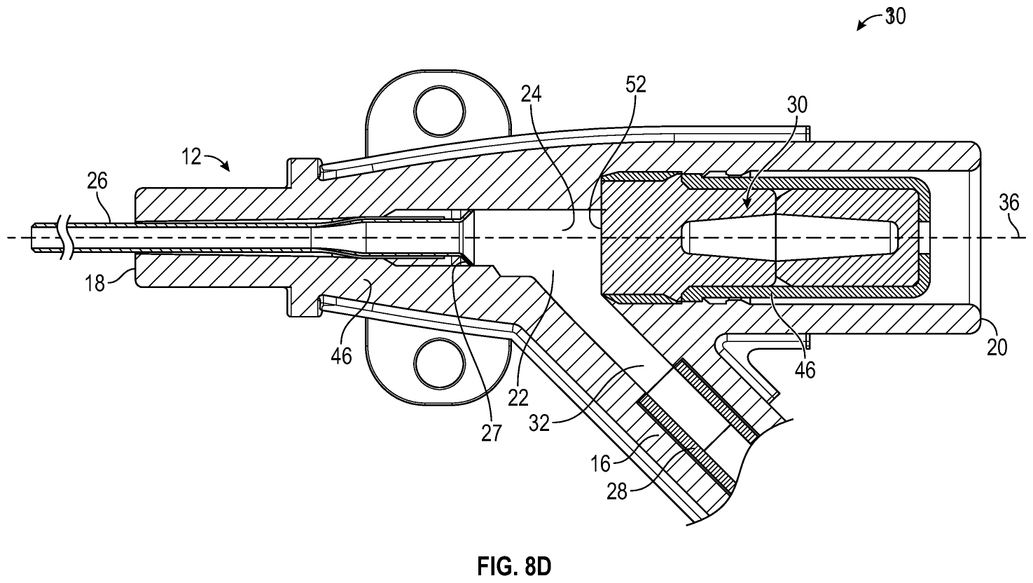

Referring now to FIGS. 8A-8C, in some embodiments, the catheter assembly 10 may decrease a volume and/or change a geometry of the region within the catheter assembly 10 that may otherwise be difficult to flush. In some embodiments, the portion of the inner surface 22 proximal to the catheter 26 and distal to the septum 30 may include a protrusion 68 extending inwardly into the lumen 24 opposite the side port pathway 32.

As illustrated, for example, in FIGS. 8A-8B, in some embodiments, the protrusion 68 may include a ramp 70 in a proximal direction. In these embodiments, the inner surface 22 may be tapered inwardly in the proximal direction to form the ramp 70. In some embodiments, a proximal end 72 of the ramp 70 may be proximate the septum 30. In some embodiments, the catheter assembly 10 may include the wedge 27 securing the catheter 26 within the distal end 18 of the catheter adapter 12. In some embodiments, a distal end 74 of the protrusion 68 may be proximate a proximal end 66 of the wedge 27.

In some embodiments, the proximal end 72 of the ramp 70 may be aligned with a wall of the catheter 47. For example, the proximal end 72 of the ramp 70 may be aligned with an axis 76 extending from an inner surface of the wall of the catheter 47, which may be spaced apart from the longitudinal axis 36 of the catheter assembly 10. Thus, in some embodiments, the protrusion 68 may decrease a volume of the lumen 24 while not increasing friction with the introducer needle that may be movable with respect to the septum 30 and the catheter 47. In some embodiments, the decrease in volume of the lumen 24 may decrease stagnant fluid within the lumen 24 distal and/or proximate to the septum 30.

In some embodiments, the proximal end 72 of the ramp 70 may be proximate the septum 30. As illustrated, for example, in FIG. 8B, in some embodiments, the distal face 52 of the septum 30 may include the protrusion 54. In some embodiments, the proximal end 72 of the ramp 70 may contact the protrusion 54. In some embodiments, the proximal end 72 may contact a distalmost surface of the protrusion 54 to reduce any cracks or grooves in which fluid may otherwise get caught.

Referring now to FIG. 8C, in some embodiments, a distal portion 82 of the protrusion 68 may be parallel to the longitudinal axis 36 of the catheter assembly 10. In some embodiments, a proximal portion 84 of the protrusion 68 may include a ramp 86 in a distal direction. In these embodiments, the proximal portion 84 may be tapered inwardly in the distal direction. In these and other embodiments, a proximal end 88 of the catheter 26 may be bonded with the catheter adapter 12. In some embodiments, elimination of the wedge 27 may allow a geometry of the catheter adapter 12 to be more filled in, reducing a volume in which fluid may become stagnant. In some embodiments, the distal portion 82 may be aligned with the wall of the catheter 26 and the axis 76.

Referring now to FIG. 8D, in some embodiments, the distal face 52 of the septum 30 may be aligned with or distal to the side port pathway 32. In some embodiments, placement of the distal face 52 of the septum 30 to be aligned with or distal to the side port pathway 32 may reduce a volume of the region proximate and/or distal to the septum 30 in which fluid may become stagnant despite flushing. In some embodiments, the septum 30 may include the elastomeric body 44 and/or the canister 46. In some embodiments, the septum 30 may include a one-piece septum, a two-piece septum, or any suitable septum.

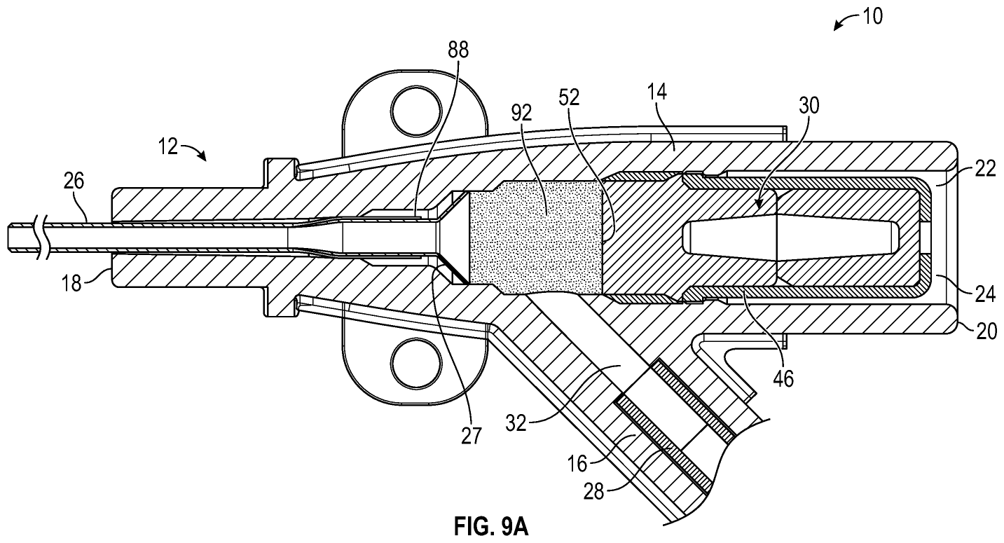

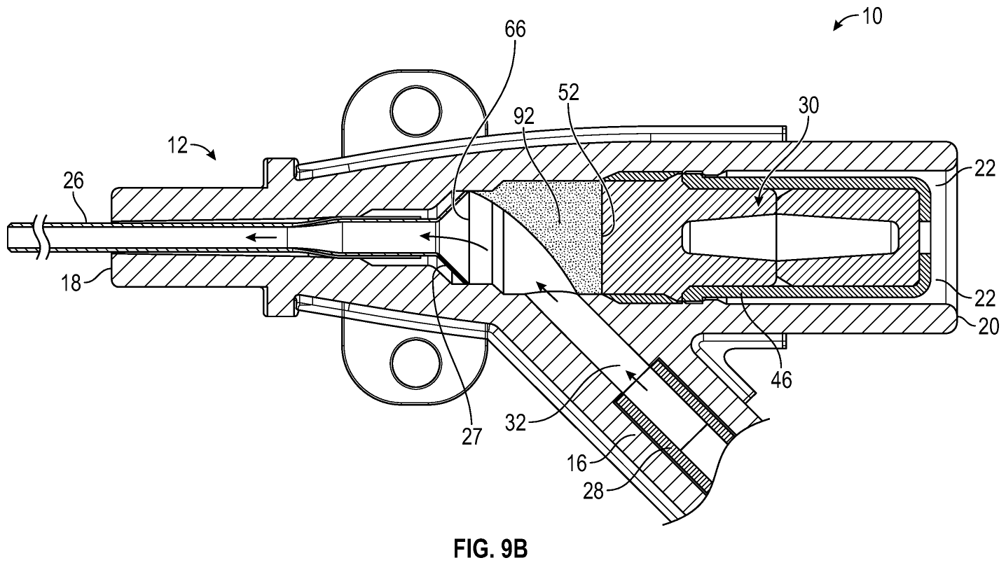

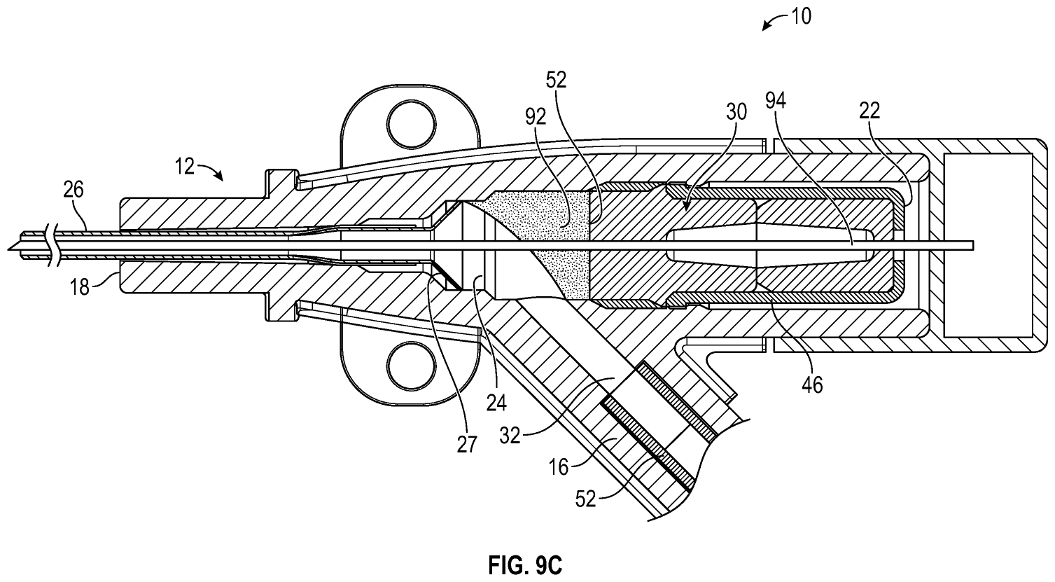

Referring now to FIGS. 9A-9C, in some embodiments, a method may include injecting a curable material 92 into the catheter adapter 12 of the catheter assembly 10. In some embodiments, the curable material 92 may be injected between a proximal end 88 of the catheter 26 and the distal face 52 of the septum 30, as illustrated, for example, in FIG. 9A. As illustrated, for example, in FIG. 9B, in some embodiments, the method may include creating a fluid pathway through the curable material 92, which may include flushing the catheter assembly 10 through the side port pathway 32. In some embodiments, the curable material 92 may include silicone, foam, gel, or another suitable material.

As illustrated, for example, in FIG. 9C, in some embodiments, the method may include inserting an introducer needle 94 through the curable material 92. In some embodiments, the method may include curing the curable material 92 after creating the fluid pathway and after inserting the introducer needle 94 through the curable material 92. In some embodiments, the introducer needle 94 may be inserted through the curable material 92 before or after creating the fluid pathway through the curable material 92.

In some embodiments, the method may include curing the curable material 92 after creating the fluid pathway and inserting the introducer needle 94 through the curable material 92. In some embodiments, the curable material 92 may be cured via heat, ultraviolet light, or another external energy source. In some embodiments, the curable material 92 may include silicone, foam, gel, or another suitable curable material.

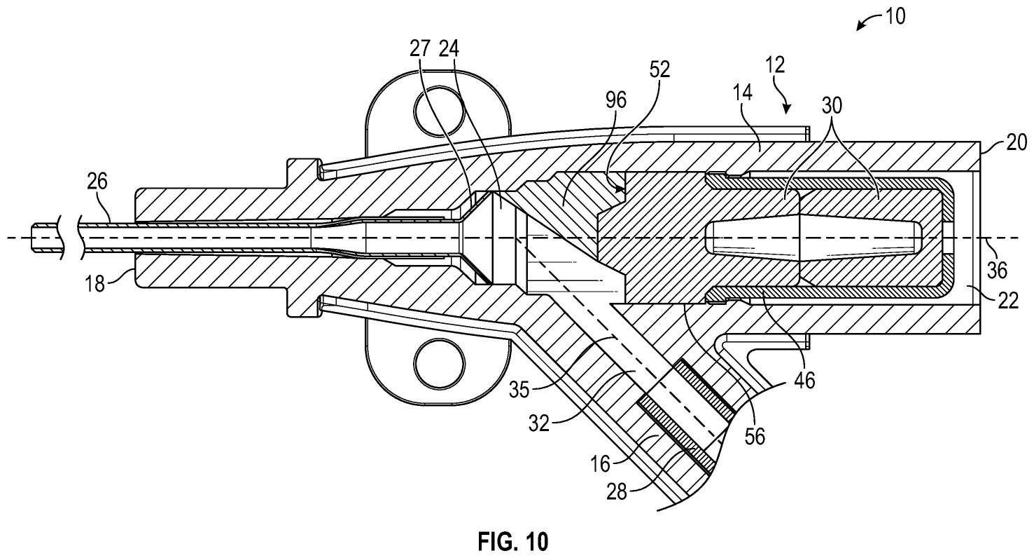

Referring now to FIG. 10, in some embodiments, an asymmetric insert 96 may be inserted within the lumen 24 between the wedge 27 and the septum 30. In some embodiments, the asymmetric insert 96 may contact the distal face 52 of the septum 30 and may reduce a volume of the region proximate and/or distal to the septum 30 in which fluid may otherwise become stagnant. In some embodiments, the asymmetric insert 96 may be constructed of a rigid, semi-rigid, or flexible material. In some embodiments, a distal face of the asymmetric insert 96 may be angled with respect to the longitudinal axis 36. In some embodiments, the distal face of the asymmetric insert 96 may be generally planar and may be angled such that a width of the asymmetric insert 96 decreases in a distal direction, facilitating fluid flow from the side port 16 into the catheter 26.

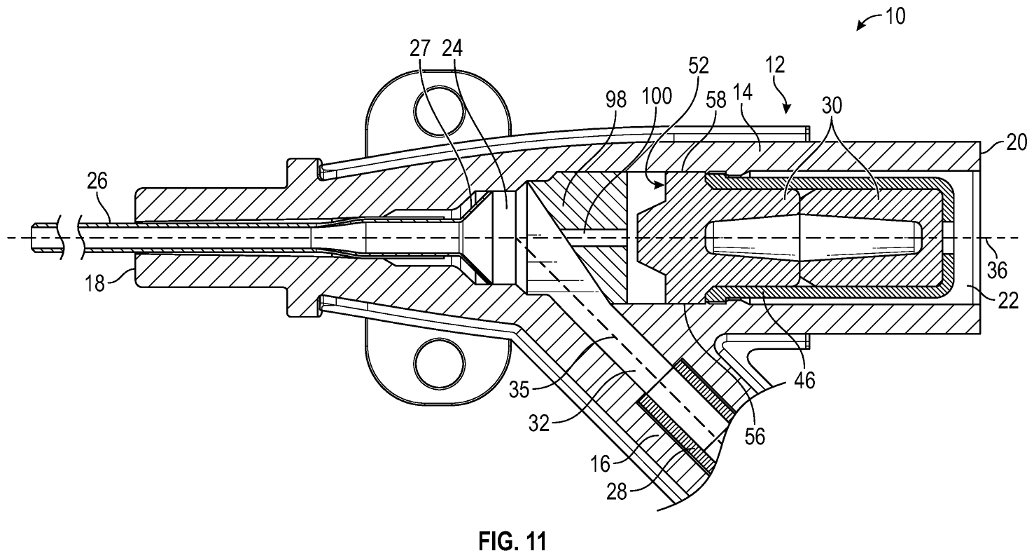

In some embodiments, the asymmetric insert 96 may include an opening therethrough similar to the insert 98 of FIG. 11. In some embodiments, the opening may be aligned with the longitudinal axis 36, which may facilitate passage of the introducer needle. In other embodiments, the asymmetric insert 96 may not cross the longitudinal axis 36 or may be disposed exterior to the longitudinal axis 36, which may facilitate passage of the introducer needle.

Referring now to FIG. 11, in some embodiments, an insert 98 may be inserted within the lumen 24 between the wedge 27 and the septum 30. In some embodiments, the insert 98 may be asymmetric. In some embodiments, the insert 98 may extend across an entirety of a width of the lumen 24. In some embodiments, the insert 98 may contact the distal face 52 of the septum 30 or may be spaced apart from the distal face 52 of the septum 30. In some embodiments, the insert 98 may reduce a volume of the region proximate and/or distal to the septum 30 in which fluid may otherwise become stagnant. In some embodiments, the insert 98 may be constructed of a rigid, semi-rigid, or flexible material. In some embodiments, a distal face of the insert 98 may be angled with respect to the longitudinal axis 36. In some embodiments, the distal face of the insert 98 may be generally planar and may be angled such that a width of the insert 98 decreases in a distal direction, facilitating fluid flow from the side port 16 into the catheter 26. In some embodiments, the insert 98 may include an opening 100 therethrough, which may be aligned with the longitudinal axis 36 and configured to allow passage of the introducer needle therein.

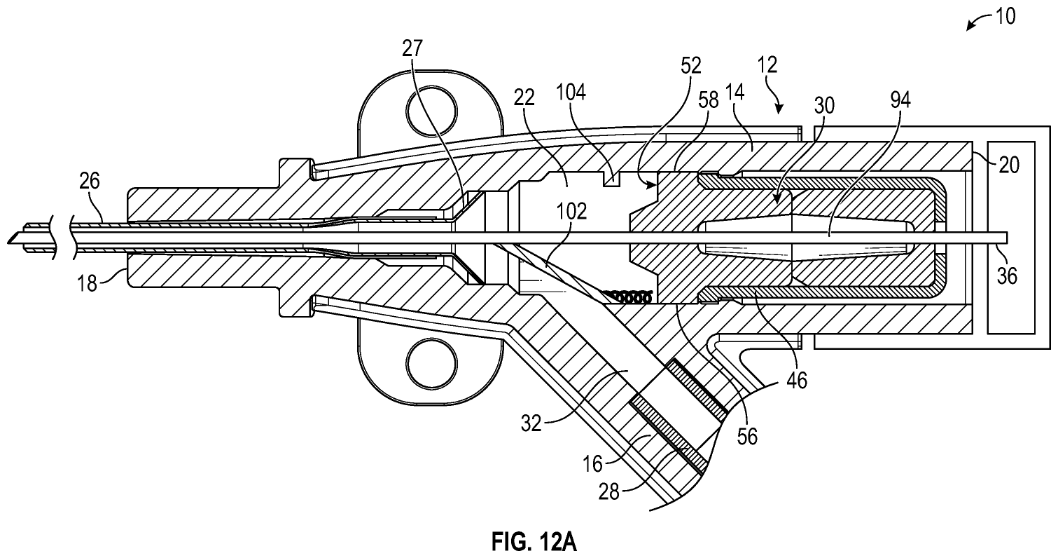

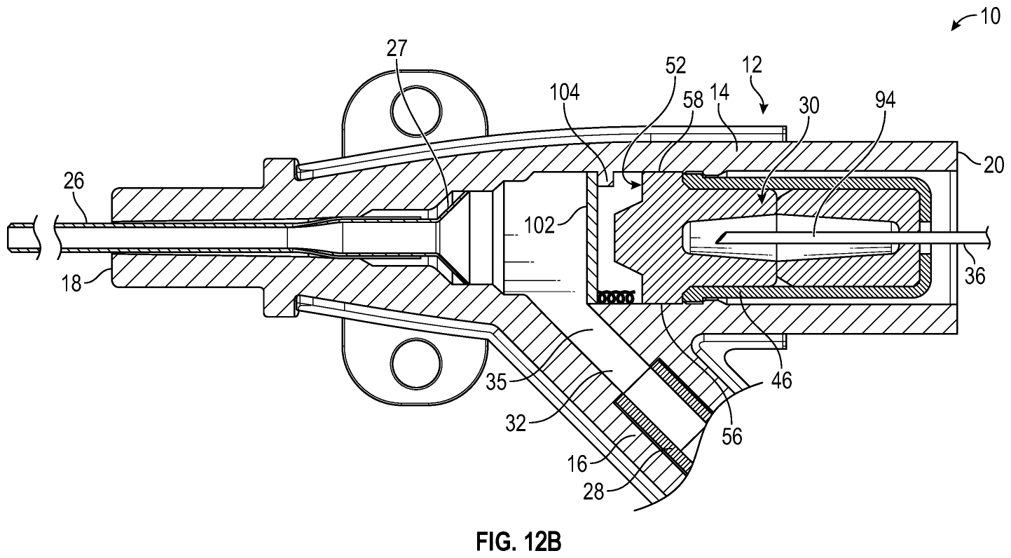

Referring now to FIGS. 12A-12B, in some embodiments, the catheter assembly 10 may include a seal component 102, which may be movable between an open position, illustrated, for example, in FIG. 12A, and a closed position, illustrated, for example, in FIG. 12B. In some embodiments, in response to the introducer needle 94 extending through the lumen 24, the introducer needle 94 may contact the seal component 102 and hold it in the open position. In some embodiments, in response to the introducer needle 94 being withdrawn proximally beyond the seal component 102, the seal component 102 may move to the closed position. In some embodiments, the seal component 102 may be generally circular or a same shape as a cross-section of the lumen 24 and may extend across an entirety of a width of the lumen 24 such that when the seal component 102 is in the closed position, fluid is prevented from entering the region proximate and/or distal to the septum 30. In some embodiments, the seal component 102 may be proximal to the side port pathway 32.

In some embodiments, a spring may be coupled to the seal component 102 and may facilitate closing of the seal component 102. In some embodiments, the inner surface 22 may include a stop 104 or protrusion that may contact the seal component 102 in the closed position and prevent passage of the seal component 102.

In some embodiments, a particular catheter assembly may include one or more features of the catheter assembly 10 of one or more of FIGS. 1-12. In some embodiments, a particular septum may include one or more features of the septum 30 of one or more of FIGS. 1-12. In some embodiments, the septum 30 of one or more of FIGS. 1-12 may include antifouling, antimicrobial, and/or antithrombogenic materials. In some embodiments, the septum 30 of one or more of FIGS. 1-12 may include a coating, which may include one or more of heparin, silver, chlorhexidine, and nitric oxide. In some embodiments, the septum 30 of one or more of FIGS. 1-12 may include a lubricious and/or fluorinated lubricant. In some embodiments, the septum 30 of one or more of FIGS. 1-12 may be molded with antifouling or antithrombogenic additives, such as, for example, polyethylene glycol (PEG), silicone, or fluorine.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter assembly, comprising:
a catheter adapter, comprising a body and a side port extending outwardly from the body, wherein the body comprises a distal end, a proximal end, and an inner surface extending through the distal end and the proximal end and forming a lumen, wherein an angle of the side port with respect to a longitudinal axis of the body is less than 45°;
a catheter extending distally from the distal end of the catheter adapter; and
a septum disposed within the lumen.

2. The catheter assembly of claim 1, wherein the septum is disposed proximal to a side port pathway extending through the side port and in fluid communication with the lumen.

3. The catheter assembly of claim 2, wherein a distal end of the septum is approximately aligned with or distal to a proximal edge of the side port pathway.

4. The catheter assembly of claim 1, wherein the angle of the side port with respect to the longitudinal axis of the body is about 25°.

5. The catheter assembly of claim 1, wherein a distal end of the septum is asymmetric.

6. A catheter assembly, comprising:
a catheter adapter, comprising a body and a side port extending outwardly from the body, wherein the body comprises a distal end, a proximal end, and an inner surface extending through the distal end and the proximal end and forming a lumen, wherein the side port comprises a side port pathway extending through the side port and in fluid communication with the lumen;
a catheter extending distally from the distal end of the catheter adapter; and
a septum disposed within the lumen, wherein a distal end of the septum is approximately aligned with or distal to a proximal edge of the side port pathway.

7. The catheter assembly of claim 6, wherein the side port pathway is generally cylindrical.

8. The catheter assembly of claim 6, wherein the distal end of the septum is aligned with the proximal edge of the side port pathway.

9. The catheter assembly of claim 6, wherein the distal end of the septum is distal to the proximal edge of the side port pathway.

10. The catheter assembly of claim 6, wherein the septum comprises a partial or complete tunnel extending therethrough and aligned with the side port pathway.

11. The catheter assembly of claim 6, wherein an angle of the side port with respect to a longitudinal axis of the body is less than 45°.

12. The catheter assembly of claim 6, wherein a distal end of the septum is asymmetric.

13. A catheter assembly, comprising:
a catheter adapter, comprising a body and a side port extending outwardly from the body, wherein the body comprises a distal end, a proximal end, and an inner surface extending through the distal end and the proximal end and forming a lumen, wherein the side port comprises a side port pathway extending through the side port and in fluid communication with the lumen;
a catheter extending distally from the distal end of the catheter adapter; and
a septum disposed within the lumen, wherein a distal end of the septum is asymmetric.

14. The catheter assembly of claim 13, wherein the distal end extends across a width of the lumen, wherein the distal end comprises a first side and a second side and is angled between the first side and the second side, wherein the first side is on a same side of the lumen as the side port, wherein the second side extends distal to the first side.

15. The catheter assembly of claim 13, wherein the distal end comprises an arm extending distally and contacting the inner surface, wherein the arm is on an opposite side of the lumen as the side port.

16. The catheter assembly of claim 15, wherein a distal end of the arm comprises an extension extending inwardly.

17. The catheter assembly of claim 16, wherein a proximal side of the extension is disposed distal to a proximal edge of the side port pathway.

18. The catheter assembly of claim 13, wherein an angle of the side port with respect to a longitudinal axis of the body is less than 45°.

19. The catheter assembly of claim 13, wherein a side of the septum closest to the side port pathway is aligned with or distal to a proximal edge of the side port pathway.

20. The catheter assembly of claim 19, wherein the side of the septum closest to the side port pathway is approximately aligned with the proximal edge of the side port pathway.
